(19) United States
(12) Patent Application Publication
(10) Pub. No.: US 2019/0224588 A1
(43) Pub. Date: Jul. 25, 2019

(54) Method for Packing Chromatography Columns

(71) Applicant: GE HEALTHCARE BIOPROCESS R&D AB, UPPSALA (SE)

(72) Inventors: Klaus Gebauer, Uppsala (SE); Spyridon Gerontas, London (GB); Jamil Shanagar, Uppsala (SE)

(21) Appl. No.: 16/306,619

(22) PCT Filed: Jun. 19, 2017
(86) PCT No.: PCT/EP2017/064876

§ 371 Date: Dec. 3, 2018

(30) Foreign Application Priority Data
Jun. 21, 2016 (GB) 1610792.2

(51) Int. Cl. B01D 15/20; B01D 15/18; G01N 30/46; G01N 30/56; G01N 30/60; A61L 2/08

(52) U.S. Cl. CPC B01D 15/206; B01D 15/1885; G01N 30/467; G01N 2030/562; G01N 30/6039; G01N 30/6043; A61L 2/081; G01N 30/56

(57) ABSTRACT

The invention discloses a method for packing a plurality of uniform chromatography columns, comprising the steps of: a) providing a plurality of chromatography columns; b) providing a plurality of chromatography resin aliquots; c) packing the chromatography resin aliquots in the chromatography columns to provide a plurality of packed chromatography columns; and d) subjecting the packed chromatography columns to repeated mechanical impacts to provide a plurality of uniform chromatography columns.

### TECHNICAL FIELD OF THE INVENTION

The present invention relates to packing of chromatography columns and in particular to packing of chromatography columns for use in processing of biopharmaceuticals. The invention also relates to methods for the manufacturing of chromatography apparatuses and in particular of chromatography apparatuses for use in processing of biopharmaceuticals.

### BACKGROUND OF THE INVENTION

Columns used in liquid chromatography typically comprise a tubular body enclosing a packed bed of porous chromatography medium through which a carrier liquid flows, with separation taking place by partitioning between the carrier liquid and solid phase of the porous medium.

Prior to any separation process, the bed has to be prepared by starting from the particulate medium that is to be introduced into the column. The process of bed formation is called ‘the packing procedure’ and a correctly packed bed is a critical factor influencing the performance of a column containing a packed bed. Typically, the packed bed is prepared by slurry packing, i.e. consolidating a suspension of discrete particles in liquid, known as slurry that is pumped, poured, or sucked into the column. Once the predetermined volume of slurry has been delivered into the column it needs to be further consolidated and compressed by moving a movable adapter down the longitudinal axis of the column towards the bottom of the column, normally at a constant speed. The excess liquid during this procedure is expelled at the column outlet, while the media particles are retained by means of a filter material, a so-called ‘bed support’, with pores too small to allow the media particles to pass though. The packing process is complete once the packed bed has been compressed by the optimum degree of compression. Another approach for column slurry packing is the flow packing method, where compression of the porous structure is primarily achieved by applying a high flow rate over the column, hereby forming a porous structure starting at the outlet bed support. The resulting drag force on the particles in the porous structure causes eventually a pressure drop and a compression of the bed. The compressed bed is finally confined by bringing the adapter into position.

The efficiency of subsequent chromatographic separation relies strongly on 1) the liquid distribution and collection system at the fluid inlet and outlet of the packed bed, 2) the special orientation (also known as the packing geometry) of the media particles in the packed bed, and 3) the compression of the packed bed. If the compression of the packed bed is too low then chromatographic separations performed on that bed suffer from “tailing” and, generally, such insufficiently compressed beds are unstable. If the compression of the packed bed is too high then chromatographic separations performed by the bed suffer from “leading” and such over-compressed beds can affect throughput and binding capacity, and, in general, give much higher operating pressures. If the compression is optimum, then the separation peaks formed during use exhibit much less leading or tailing and are substantially symmetrical. The optimum degree of compression is also crucial for achieving good long-term stability of the porous structure, hereby securing optimal performance throughout a number of process cycles. The optimum degree of compression required for a column is determined experimentally for each column size (width or diameter), bed height, and media type.

A particular issue is that it is often desirable to scale chromatographic processes by parallel coupling of several columns in order to increase capacity. The variability of current packing procedures has however been a serious obstacle, since the permeabilities and correspondingly the flow velocities will vary between the individual columns, causing excessive band broadening over the parallel assembly. Methods of dry packing of swellable media have been suggested as a remedy to this problem (see US20140224738 and US20120267299, both of which are hereby incorporated by reference in their entireties). Even with these methods some variability between the individual columns is however observed and accordingly there is a need for methods to further reduce column-to-column variability.

### SUMMARY OF THE INVENTION

One aspect of the invention is to provide a method for packing a plurality of uniform chromatography columns. This is achieved by a method comprising the steps of:

a) providing a plurality of chromatography columns;
b) providing a plurality of chromatography resin aliquots;
c) packing the chromatography resin aliquots in the chromatography columns to provide a plurality of packed chromatography columns; and
d) subjecting the packed chromatography columns to repeated mechanical impacts.

One advantage is that a high degree of column-to-column uniformity is achieved, allowing parallel connection of several chromatography columns, e.g. for scaling of a process. Further advantages are that the method is convenient and useful in a manufacturing environment.

A second aspect of the invention is a method for manufacturing a chromatography apparatus, comprising the steps of:

a) performing the method as outlined above; and
b) fluidically connecting the chromatography columns in parallel.

A third aspect of the invention is a method for manufacturing a chromatography apparatus, comprising the steps of:

a) providing a plurality of chromatography columns;
b) providing a plurality of chromatography resin aliquots;
c) packing the chromatography resin aliquots in the chromatography columns to provide a plurality of packed chromatography columns;
d) stacking and fluidically connecting the chromatography columns in parallel to form a chromatography apparatus and
e) subjecting the chromatography apparatus to repeated mechanical impacts.

Further suitable embodiments of the invention are described in the dependent claims.

### DRAWINGS

FIG. 1 shows a) a perspective view of an assembled cartridge, with the internal channel system showing and b) a side view with the internal bed cavity, bed support nets and end plugs highlighted.

FIG. 2 shows stacks of cartridges in a) parallel configuration and b) serial configuration. The flow paths are indicated by arrows. The dashed lines indicate flow paths broken by the insertion of sealing pins in the cartridges.

FIG. 3 shows the protocols used in the examples.

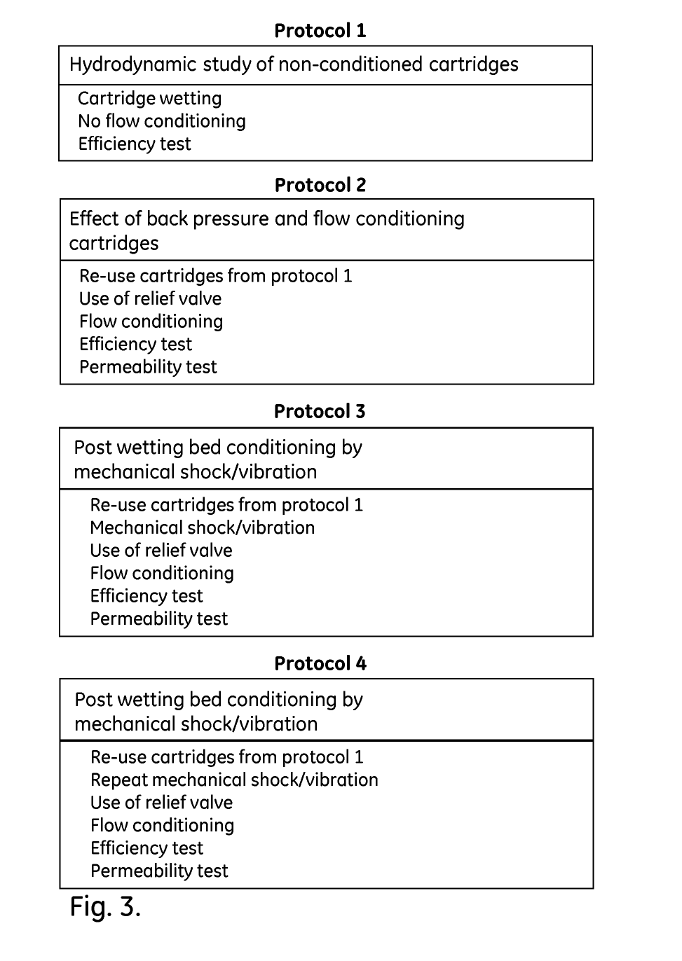

FIG. 4 shows acetone peak profiles of non-conditioned cartridges.

FIG. 5 shows the combined effect of back pressure and flow conditioning on the acetone peak profiles of the efficiency test of the cartridges.

FIG. 6 shows permeability measurements under the effect of back pressure and flow conditioning.

FIG. 7 shows the effect of post wetting bed conditioning by mechanical shock/vibration on the acetone peak profiles of the efficiency test of the cartridges.

FIG. 8 shows permeability measurements under the effect of post wetting bed conditioning by mechanical shock/vibration. The permeability of cartridge 11 was not estimated due to leakage during the application of post wetting bed conditioning by mechanical shock/vibration.

FIG. 9 shows the effect of repeated post wetting bed conditioning by mechanical shock/vibration on the acetone profiles.

FIG. 10 shows permeability measurements for cartridges 1, 2, 4, 5, 6, 8 and 9 under the effect of repeated post wetting bed conditioning by mechanical shock/vibration. Permeability was not estimated for rest of cartridges.

FIG. 11 shows acetone peak profiles of parallel assemblies of two cartridges.

FIG. 12 shows acetone peak profiles of parallel assemblies of four cartridges.

FIG. 13 shows acetone peak profiles of a cartridge before and after post wetting bed conditioning by mechanical shock and also after an additional step of ultrasound treatment.

### DEFINITIONS

To more clearly and concisely describe and point out the subject matter of the claimed invention, the following definitions are provided for specific terms that are used in the following description and the claims appended hereto.

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least each numerical parameter should at least be construed in light of the number of reported significant digits and by applying ordinary rounding techniques.

As used herein to describe the present invention, directional terms such as “up”, down”, “upwards”, “downwards”, “top”, “bottom”, “vertical”, “horizontal”, “above”, “below” as well as any other directional terms, refer to those directions in the appended drawings.

### DETAILED DESCRIPTION OF EMBODIMENTS

In one aspect, the present invention discloses a method for packing a plurality of chromatography columns. The method can provide uniform columns and comprises the steps of:

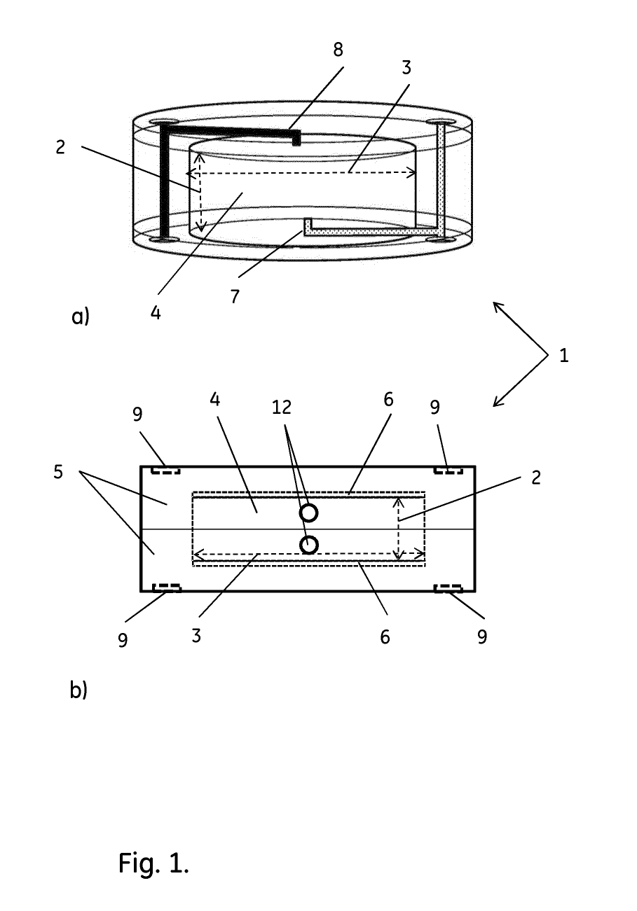

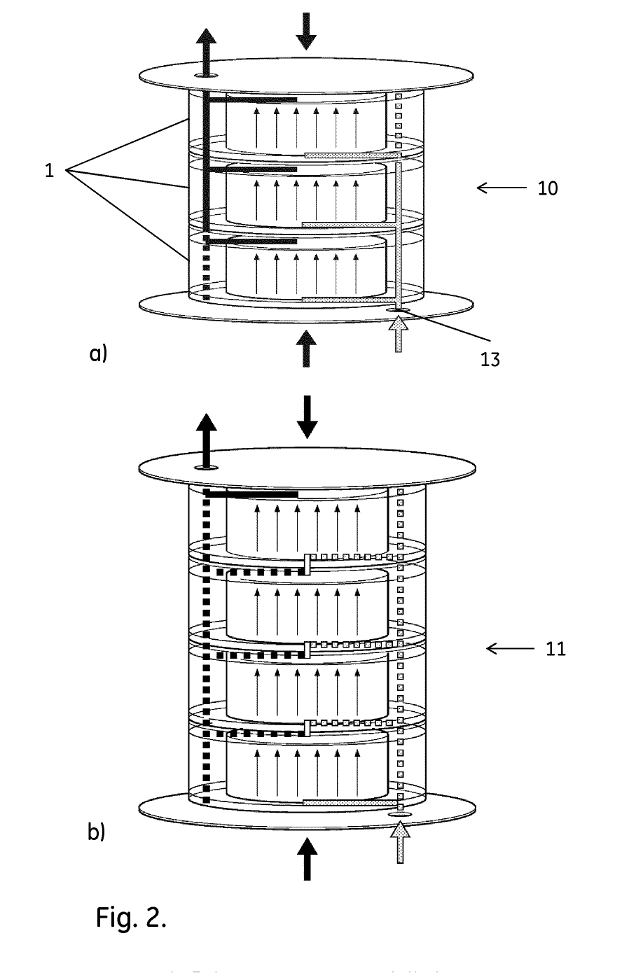

a) Providing a plurality of chromatography columns 1. The columns are suitably empty preparative columns for packed bed liquid chromatography media, e.g. columns capable of accommodating bed volumes within the range of 100 mL-25 L, such as 100 mL-10 L or 200 mL-5 L. The columns may e.g. be capable of accommodating bed heights within the range of 1-10 cm, such as 1-5 cm. They may also, or alternatively, be capable of accommodating packed beds with bed width-to-bed height ratios within the range of 2-10, such as 2-5, since high width/low height beds are desirable for parallel processing and present particular packing issues. Suitably, the chromatography columns may be substantially identical, e.g. having identical design and identical dimensions apart from variations caused by normal manufacturing tolerances (e.g. +/−1.0 mm or +/−0.5 mm). The chromatography columns may e.g. be chromatography column cartridges 1, as shown in FIG. 1, adapted to be stacked with like chromatography column cartridges and to be fluidically connected in parallel and/or serially. The stacking can be made vertically, as shown in FIG. 2, but it can equally well be done horizontally and it is also possible to have different cartridge geometries, e.g. rectangular/quadratic cartridges. Suitably, the chromatography columns or cartridges may be single use columns/cartridges, e.g. constructed from plastics. They may also be capable of withstanding sterilization, e.g. by gamma irradiation. The chromatography columns can be fixed volume chromatography columns, e.g. with fixed bed volume and bed height. The packed bed can e.g. be delimited by fixed distribution nets 6 and a fixed side wall or side wall components 5. With this arrangement, the columns may be devoid of movable pistons.
b) Providing a plurality of chromatography resin aliquots. The chromatography resin can be a swellable resin, such as water-swellable resin (e.g. a crosslinked polysaccharide resin or other hydroxyfunctional polymer resin), and the aliquots may be prepared from dry chromatography resin, e.g. by weighing, which allows a high degree of precision and thus a high degree of uniformity between the aliquots. The aliquots may be substantially identical, e.g. differing from each other by less than 5.0 percent with respect to dry weight, such as by less than 2.0 percent or by less than 1.0 percent. The chromatography resin used to prepare the aliquots may e.g. be a single batch or pool of dry chromatography resin. Alternatively, the aliquots may be prepared from different batches or pools, if the batch-to-batch or pool-to-pool variability with respect to particle size distribution and/or degree of swelling is low enough not to affect the column uniformity significantly. The aliquots may either be used in dry form in the subsequent steps or they may be reswollen with a liquid, e.g. an aqueous liquid, and used in wet or slurry form in the subsequent steps.
c) Packing the chromatography resin aliquots in the chromatography columns to provide a plurality of packed chromatography columns. If the resin aliquots from step b) are dry, the packing may involve transfer of each aliquot to a column, closing the column and reswelling the resin with a liquid, e.g. an aqueous liquid. The size of the aliquots may then be chosen such that the swollen volume slightly exceeds the column volume (the swollen volume may e.g. be 105-120% of the column volume), such that the swelling leads to a suitable degree of column compression. If the aliquots from step b) are wet or reswollen, each aliquot may be transferred as a slurry to a column and packed by standard methods for wet packing of chromatography resins. It is also possible to use an intermediate method, where dry gel is transferred to the column and reswollen in the column before closing the column. In the two latter cases, the column may be compressed to a suitable degree by a movable column adaptor. The packing may e.g. be performed according to the methods described in US20120267299 or US20140224738, which are hereby incorporated by reference in their entireties.
d) Subjecting the packed chromatography columns to repeated mechanical impacts. This step improves the column-to-column uniformity considerably. The number of impacts may be 5 or higher, such as at least 10, at least 20 or at least 30. The impacts may be caused by relative motion between the column and an object, such as e.g. by moving the column towards a stationary object or by moving an object towards a stationary column. The impacts may be directly between the column and the object, but they may also be between the object and an energy-transfer object in direct contact with the column, e.g. a piece of rigid material (e.g. metal or rigid plastic) contacting the column. Suitably, the impacts may be evenly distributed along the column perimeters, e.g. by turning the column between the impacts, moving an impacting object or by using several impacting objects. The kinetic energy of individual impacts may e.g. be 0.1-100 J, such as 0.1-50 J or 1-25 J, or 0.05-50 J per kg column weight and/or the integral kinetic energy of the entire treatment process for each column may e.g. be 10-5000 J, such as 10-2000 J or 500-2000 J or 5-2000 J per kg column weight. For determining the kinetic energy (Eₖᵢₙ), the formula Eₖᵢₙ=m*v²/2 may be used, where v is the relative velocity between the column and the object at impact and m is the mass of the moving item (the column or the object, if one of them is stationary). The impacts may be in the form of individual impacts or in the form of vibration, e.g. caused by attaching one or more vibrators to the columns. A vibrator will comprise a moving (vibrating) object, impacting the column either directly or via an energy-transfer object. The vibration frequency can be low (e.g. 1-200 Hz, such as 100-200 Hz) or higher (e.g. up to 100 kHz, such as 200 Hz-100 kHz or 20-50 kHz). It can be advantageous to use ultrasound treatment (e.g. 20-100 kHz, such as 20-50 kHz), either in step d) or as a separate further step after conditioning with individual impacts or low frequency vibration. As the number of impacts caused by a vibrator will generally be high (e.g. at least 100 or at least 1000), the kinetic energy per impact may in this case be lower (e.g. 1 mJ-1 J per impact). The effect of the mechanical impact treatment is to improve the column-to-column uniformity. This can be measured e.g. by the hydraulic permeability, the retention volume for a non-binding species, the plate height or plate volume for a non-binding species or by the breakthrough capacity (e.g. the 10% breakthrough capacity) for a binding species. Typically, the column-to-column differences will be: hydraulic permeability—less than 1*10⁻⁸ cm², such as less than 5*10⁻⁹ cm², or expressed in relative terms—less than 50%, such as less than 25% or less than 10%; retention volume—less than 0.1 column volumes, such as less than 0.05 column volumes; plate height—less than 200 micrometers, such as less than 100 micrometers. The columns can suitably be subjected to the mechanical impacts in a separate step, after the packing step c).

In some embodiments, the packed chromatography columns are radiation sterilized. This may be achieved by subjecting them to radiation sterilization, e.g. by gamma irradiation, during or after step c), or alternatively after step d).

In a second aspect, the invention discloses a method for manufacturing a chromatography apparatus 10, comprising the steps of:

a) performing the packing method as discussed above; and
b) fluidically connecting the plurality of chromatography columns in parallel. The connection may e.g. comprise fluidically connecting the plurality of columns to a single sample inlet 13, which allows for parallel processing of the sample. The connection may be achieved simply by tubing, but it may also be achieved by stacking the columns or column cartridges, wherein conduits in the columns or cartridges are fluidically connected to form the parallel connection.

The stack can then suitably have a single inlet and a single outlet, both of which have branches to/from the individual columns/cartridges. Examples of suitable stacking modes are shown in US201330068671 and US20140263012, which are hereby incorporated by reference in their entireties.

More specifically, the manufacturing method may comprise the steps of:

a) providing a plurality of chromatography columns 1;
b) providing a plurality of chromatography resin aliquots;
c) packing the chromatography resin aliquots in the chromatography columns to provide a plurality of packed chromatography columns;
d) stacking and fluidically connecting the chromatography columns in parallel to form a chromatography apparatus 10 and
e) subjecting the chromatography apparatus to repeated mechanical impacts.

The method may further comprise subjecting the chromatography apparatus to radiation sterilization after step d), such as before step e).

### Example 1

### Modular Chromatography Cartridges (See FIGS. 1 and 2)

The cartridges 1 were manufactured by GE Healthcare, Uppsala, Sweden and they were pre-filled with dried resin. The cartridges had a cassette format, which allows stacking one of them on top of each other. Each cartridge had a bed height 2 of 30 mm and an internal (bed) diameter 3 of 128.6 mm, yielding a packed bed volume of 390 ml in a bed cavity 4. It was assembled by aligning two identical parts 5 with a rubber seal in between them and screwed tightly with a total of eight M6 screws. The end of the bed cavity portion in each part was fitted with a coarse distribution net 6 underneath a bed support net with an average pore size of 23 μm and communicating with an inlet and an outlet channel 7,8 respectively. The pressure drop limit for the cartridge was 3 bar.

The operation of a single cartridge required the installation of two seal pins inserted via openings 9 to block the unused pathway. The seal pins had to be inserted in such a way so as not to cover the inlet and outlet hole of the cartridge. The inlet and outlet holes were then fitted with O-rings and the cartridge was mounted in the stand 10,11. For multiple cartridge configurations, the stacked cartridges can then—depending on the configuration of end plugs—either be run in a parallel 10 or a serial 11 setup (FIGS. 2a and b respectively). Large O-rings need to be fitted on the side where liquid flows between the cartridges. When operating in parallel setup, two seal pins were installed in different direction for each cartridge to generate a pathway as shown by the arrows in FIG. 2a. The one on the inlet side was fitted to the top column, facing down towards the inlet and the one on the outlet was fitted on the bottom column facing upwards (FIG. 2a). If more than two cartridges are stacked together, the middle cartridges need to be fitted with O-rings without using any stop plugs to allow liquid to flow through.

### Chromatography Resin

Strong anion Capto Q™ resins (GE Healthcare, Uppsala, Sweden) were used in this study. It is an agarose based anion exchange chromatography resin with an average particle size of 90 μm. The functional group of Capto Q is −N⁺(CH₃)₃. The lot number of the resin used in this study was 10134514. The resin was dried according to the following method:

A glass filter funnel was filled to approx. 60% with slurred gel and connected to a vacuum. Initially the gel was washed with purified water at a relatively fast flow rate. Then followed by ethanol at a lower rate to give approx. 15-20 min residence time.

The gel was then washed with acetone at the same flow speed as the ethanol. The gel was not allowed to run dry initially. After adding a final portion of acetone, the gel was allowed to run almost dry before being transferred to polypropylene beakers. The beakers were covered with a paper cloth and placed in a vacuum oven for drying under vacuum for 3-7 days. The drying was performed at room temperature.

### Chromatography System

An ÄKTApilot™ system (GE Healthcare, Uppsala, Sweden) was used for cartridge efficiency test and permeability measurements. The tubing used for testing was kept as short as possible with a minimal inner diameter without creating an excessive pressure drop. The dead volume of the ÄKTApilot chromatography system was 22.6 ml.

### Swelling/Wetting Method

The cartridge was filled with dry resin from the two holes 12 (sealed with screws) in its lateral side (FIG. 1b). The resin was weighed in with the cartridge on a balance and the amount was calculated to give a swollen volume of 1.10*390 ml, according to the liquid uptake determined in a separate swellability test. Then, the cartridge was shaken slightly to mix the dry resin and the cartridge was mounted in a holder. The amount of resin to be added in the cartridge depends on resin's liquid uptake. This was measured according to:

Approx. 20 g of dried gel was weighted into a measuring cylinder (250 ml) and then water was added to approx. 200 ml. The gel was then suspended by hand; placing one hand on top of the cylinder which had first been sealed with a piece of Parafilm over the opening and then shaking up and down and/or sideways until all dry gel was dispersed in the water. The slurry was then left standing over night. On the following morning the volume of the sedimented gel was read on the measuring cylinder. By correlating the volume to the weight of the gel, the swelling factor was determined and expressed in ml/g. For example 20 gram of gel resulting in 100 ml sedimented gel will give a swelling factor of 100/20=5 ml/g.

Ethanol 20% v/v in reverse osmosis (RO) water was pumped into the cartridge upwards to wet the dried resin particles. Ethanol was used in order to lower the surface tension of the water and flush trapped air at a faster rate. The liquid flowed from the bottom across the dry particle bed. The rate of liquid addition to the dry swellable particles was kept low in order not to exceed the rate of capillary suction by the particles. The rate of capillary suction can be defined as the rate of liquid moving across the bed by optical, gravimetric from the lower end of a column with dry swellable particles. During wetting, the dry resin particles absorb liquid and start swelling. The volume of resin inside the cartridge starts increasing covering the whole internal space of the cartridge. Then, compression of resin takes place as there is no free space within the cartridge for the resin particles to cover. The compression level though was kept within the typical column operation limits suggested by the manufacturer. A linear velocity of 30 cm h⁻¹ for three column volumes was used for all 11 individual cartridges during the addition of 20% v/v ethanol in RO water to the dry particles, following by 5 column volume of RO water to rinse the beads after swelling. The running conditions for swelling/wetting of cartridges are shown in Table 1.

TABLE 1
Swelling/wetting method

Step 1: Fill cartridge with dried resin according to dried resin's liquid
uptake
Step 2: Shake cartridge to mix resin and mount cartridge to the holder
Step 3: Swelling/wetting of resin

| | |
|---|---|
| Inlet: | 20% v/v ethanol in RO water |
| Outlet: | waste |
| Airtrap: | bypass |
| Cartridge: | Inline, running upwards at 30 cm/h |
| Volume: | 3 CV |

Post wetting cartridge conditioning

Step 1: Equilibrate

| | |
|---|---|
| Inlet: | RO water |
| Outlet: | waste |
| Airtrap: | bypass |
| Cartridge: | Inline, running upwards at 30 cm/h |
| Volume: | 5 CV |

### Cartridge(s) Efficiency Test

The column efficiency can be determined by the reduced plate height, derived from the plate number and height equivalent to a theoretical plate according to the following equations⁹:

$$\begin{matrix}N = 5.54 {( \frac{{V}_{R}}{{W}_{h}} )}^{2} & ( 1 ) \\ HETP = \frac{L}{N} & ( 2 ) \\ h = \frac{HETP}{{d}_{p}} & ( 3 )\end{matrix}$$

where N is the plate number, V_{R} is the retention volume, Wₕ is the peak width at half peak height, HETP is the height equivalent of a theoretical plate, L is the bed height, N is the plate number, h is the reduced plate number and dₚ is the particle diameter.

A small molecule non-retained species, acetone 2% v/v in RO water, was applied as tracer with a volume of 1.5% of cartridge internal volume to analyse the residence time distribution at the cartridge outlet. The residence time distribution should be represented as an ideal Gaussian peak shape. The absorbance of the tracer was detected at 280 nm. The linear velocity was 30 cm h⁻¹ (65 ml min⁻¹). The peak asymmetry can be determined by the asymmetry factor Aₛ, a ratio between the distance from the leading edge of a peak to the centre of the peak and the distance from the peak centre to the trailing edge. The asymmetry factor Aₛ is calculated from the peak width at 10% of its height:

$${A}_{s} = \frac{b}{a} \qquad ( 4 )$$

where a is the distance from the leading edge of the peak to the midpoint of the peak and b is the distance from the midpoint of the peak to the trailing edge.

The conditions of the efficiency test were kept as constant as possible in order to achieve comparable results, since changes in buffers, sample volume, liquid velocities, liquid pathway, temperature, etc. will influence the result. The running conditions for efficiency tests are shown in Table 2.

TABLE 2
Running conditions for efficiency tests
Running conditions

| | |
|---|---|
| Sample: | 2% acetone (v/v) in RO water |
| Eluent: | RO water |
| Liquid velocity: | 30 cm/h (flow directed upwards) |

Running method

Step 1. Equilibrate

| | |
|---|---|
| Inlet: | RO water |
| Outlet: | waste |
| Airtrap: | bypass |
| Column: | inline, running upwards |
| Volume: | 3 CV |

Step 2. Apply sample

| | |
|---|---|
| Inlet: | sample |
| Outlet: | waste |
| Airtrap: | bypass |
| Column: | inline, running upwards |
| Volume: | 1.5% of CV |

Step 3. Elute sample

| | |
|---|---|
| Inlet: | RO water |
| Outlet: | waste |
| Airtrap: | bypass |
| Column: | inline, running upwards |
| Volume: | 2 CV |

### Post Wetting Bed Conditioning by Mechanical Shock/Vibration

The process of post wetting bed conditioning of the cartridge by mechanical shock/vibration involved three steps. In the first step, the screws of the cartridge were tightened but not over torqued (before getting snug-tightened). The seal pins were removed and all openings were sealed with plastic caps. In the second step, the cartridge was knocked across its lateral side onto a hard surface 42 times during 2 min, with rotation between the knocks so that the knocks were evenly distributed along the cartridge perimeter, and it was placed in the holder. The weight of the packed cartridge was approx. 2.5 kg and the velocity at impact was approximately 3 m/s, giving a kinetic energy of 11 J per knock and 460 J for the entire knocking sequence. In the last step, the cartridge was flow conditioned with 10 CV of RO water pumped at 600 ml min⁻¹ downwards and then upwards (same flow rate).

The packed resin in the cartridges was disrupted in order to achieve a more homogenous packed bed structure than the one obtained after completing the wetting of the dried resin. The effectiveness of the post wetting bed conditioning by mechanical shock/vibration was tested by conducting efficiency tests and by measuring the permeability of each of the cartridges. The process was repeated till the permeability of each cartridge was within 10% of the average permeability of the rest of the eleven cartridges and the asymmetry factor was within the range required when using typical chromatography columns (0.8-1.8).

### Permeability Measurements

The permeability was determined by a pressure-flow measurement where cartridge pressure drop was monitored when increasing flow rate manually at internals. More specifically, the pressure drop was recorded between two Digitron 2083P7 pressure meters (Digitron, Devon, UK) placed at the inlet and outlet of each cartridge using T-junctions (i.d. 9.4 mm (0.37 in), 3 TC; GE Healthcare, Uppsala, Sweden). The flow rate was increased incrementally from 100 ml min⁻¹ to 800 ml min⁻¹ in fixed steps of 100 ml min⁻¹. Each step lasted 3 min, which period the pressure drop stabilised. The permeability was calculated from Darcy's Law, which is expressed mathematically by the following equations:

$$\begin{matrix}Q = - \frac{kA Δ P}{μ L} & ( 5 ) \\ k = - \frac{μ LQ}{A Δ P} & ( 6 )\end{matrix}$$

where Q is the flow rate, A is the cross sectional area of the packed bed, L is the length of the packed bed, μ is the dynamic viscosity of the mobile phase, P is pressure and k is the permeability.

### Results and Discussion

Four protocols were constructed in order to enhance cartridge performance and achieve similar performance with the one of typical chromatography columns in terms of efficiency test and permeability measurements. The first protocol shows the results from efficiency test of cartridges without conducting any pre-treatment after wetting the dried resin. The dried resin was wetted only once, while the different post wetting treatments have been applied subsequently. The second protocol demonstrates the combined effect of back pressure and flow conditioning. The third protocol shows the effect of post wetting bed conditioning by mechanical shock/vibration on the efficiency test and permeability measurements and the forth protocol investigates the effect of repeated post wetting bed conditioning by mechanical shock/vibration till permeability becomes consistent across all cartridges. Efficiency tests for parallel cartridge assemblies of two and four cartridges have been conducted to verify the effectiveness of the post wetting bed conditioning by mechanical shock/vibration.

### Hydrodynamic Study of Non-Conditioned Cartridges

The cartridges filled with dried resin were wetted as described above. The efficiency test for each cartridge was conducted at a linear velocity of 30 cm h⁻¹ (65 ml min⁻¹) with the flow being directed upwards. The screws of the cartridges were not tightened further. No flow conditioning (flow at high flow rate) was conducted after the wetting of the dried resin encased in the cartridges and no relief valve was placed downstream in order to create back pressure.

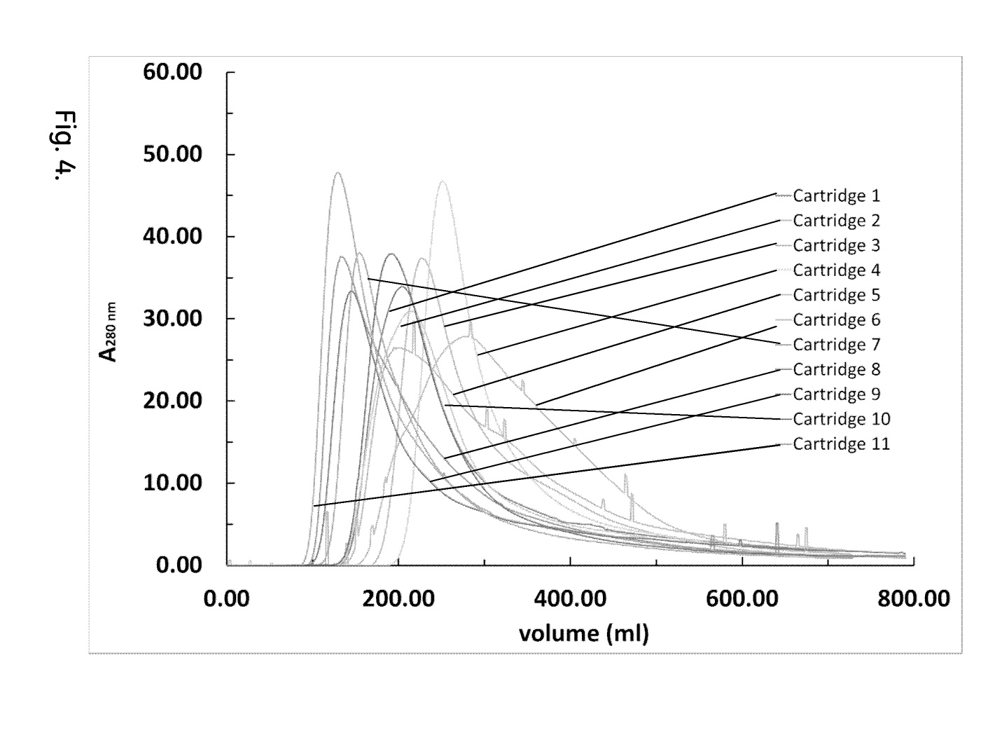

FIG. 4 shows the acetone profile for each of the eleven cartridges. The acetone profiles are not consistent. This shows that the packing of the resin inside the cartridges was not homogenous highlighting the need for developing protocols for cartridge pre-treatment before purification use. Table 3 shows the main metrics of the efficiency test. The main peak of all acetone profiles appears at much lower eluent volume than 390 ml, which is the volume of the packed bed. This may be the result of channeling or loose packing in the centre of the cartridge, which allows the acetone to be eluted at higher linear velocities and consequently to have lower residence time. All acetone profiles show extensive tailing, which is indicative of over compression of the packed resin at the internal walls of the cartridges. This tailing is not consistent and varies across all cartridges. Additionally, the acetone profiles of three cartridges have several small peaks. This is indication that there was air still trapped in the cartridges after the process of wetting the dried resin.

TABLE 3
Metrics of non-conditioned cartridges

| | Volume at | Peak width at | Asymmetry | | HETP |
|---|---|---|---|---|---|
| Cartridge ID | peak (ml) | 50% (ml) | (10% peak) | N | (μm) |
| 1 | 204.2 | 17.0 | 5.4 | 804 | 37.3 |
| 2 | 214.2 | 15.5 | 4.2 | 1063 | 28.2 |
| 3 | 226.7 | 18.7 | 6.3 | 818 | 36.7 |
| 4 | 251.0 | 23.4 | 4.9 | 640 | 46.9 |
| 5 | 200.7 | 13.2 | 8.5 | 1278 | 23.5 |
| 6 | 283.0 | 14.8 | 2.5 | 2021 | 14.8 |
| 7 | 154.3 | 19.0 | 8.6 | 365 | 82.1 |
| 8 | 135.9 | 18.7 | 9.5 | 293 | 102.2 |
| 9 | 145.2 | 16.7 | 9.4 | 419 | 71.6 |
| 10 | 192.9 | 19.0 | 4.4 | 574 | 52.2 |
| 11 | 129.5 | 23.9 | 6.6 | 163 | 184.3 |

### Effect of Back Pressure and Flow Conditioning on the Hydrodynamic Behaviour of Cartridges

The second protocol was based on the combined effect of back pressure and flow conditioning after wetting of the dried resin. Specifically, following wetting of the dried resin during hydrodynamic study of non-conditioned cartridges (section 3.1), the cartridges underwent flow conditioning with 10CV with flow directed downwards and then upwards at 800 ml min⁻¹ in order to improve the distribution of the resin encased in the cartridges. Then, a 3 bar back pressure was applied downstream during the efficiency tests and the permeability measurements. The efficiency test for each cartridge was conducted at a linear velocity of 30 cm h⁻¹ (65 ml min⁻¹) with the flow being directed upwards. The screws of the cartridges were snug-tightened before placing the cartridge in the holder. The permeability of the resin in the cartridges was estimated according to the experimental test protocol provided above.

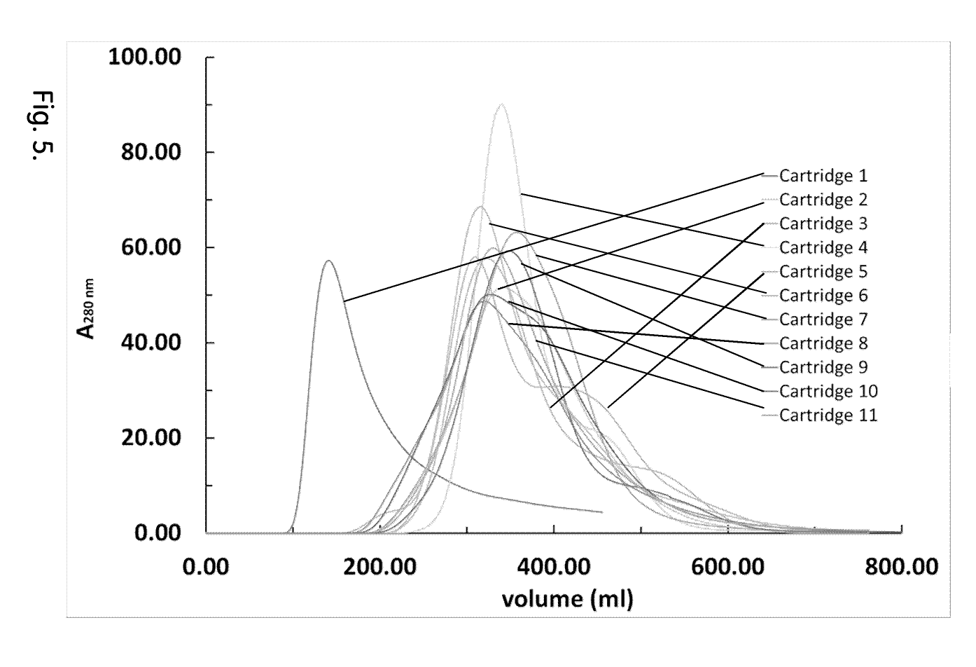

FIG. 5 presents the acetone efficiency tests under the combined effect of back pressure and flow conditioning. The acetone profiles were more consistent than the acetone profiles obtained when the cartridges had not conditioned. The peak of the acetone profiles appeared closer to the volume of the packed bed of the cartridge, which is an indication that the bed is more homogenous. Nevertheless, they were still not overlapping and some of them had secondary peaks. The modified protocol in running the cartridges seemed to have no effect on cartridge 1. Possibly, the pressure drop during flow conditioning was not enough to improve the packed bed structure. The metrics of the efficiency tests were closer together, but the deviation between them was still wide. For example the asymmetry factor varied from 1.1 to 7 when ideally it should be close to 1 (Table 4).

TABLE 4
Metrics of the efficiency test under the effect of back pressure and flow
conditioning

| | Volume at | Peak width at | Asymmetry | | HETP |
|---|---|---|---|---|---|
| Cartridge ID | peak (ml) | 50% (ml) | (10% peak) | N | (μm) |
| 1 | 141.3 | 154.0 | 7.0 | 4.7 | 6430 |
| 2 | 342.5 | 149.0 | 1.6 | 29.3 | 1024 |
| 3 | 325.7 | 112.0 | 3.1 | 46.9 | 640 |
| 4 | 337.0 | 77.0 | 2.8 | 106.1 | 283 |
| 5 | 313.0 | 104.0 | 3.3 | 50.2 | 598 |
| 6 | 316.0 | 153.0 | 3.0 | 23.6 | 1269 |
| 7 | 357.0 | 140.0 | 1.1 | 36.0 | 833 |
| 8 | 322.0 | 160.0 | 1.8 | 22.4 | 1337 |
| 9 | 349.0 | 115.0 | 2.1 | 51.0 | 588 |
| 10 | 330.7 | 185.0 | 1.7 | 17.7 | 1695 |
| 11 | 330.0 | 120.0 | 2.0 | 41.9 | 716 |

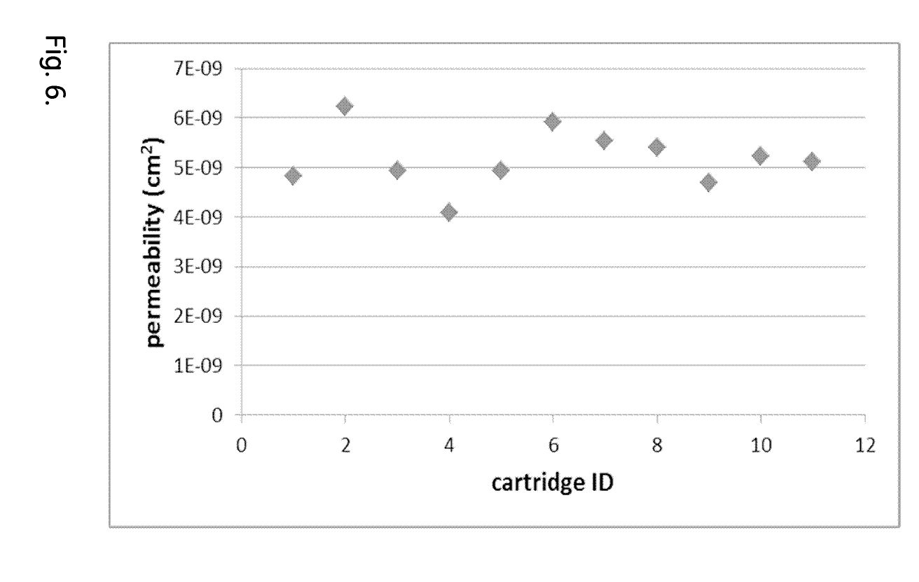

The permeability measurements targeted to estimate how much pressure is needed to achieve a specific flow rate. It is another metric of the homogeneity of the resin packing. FIG. 6 shows the permeability measurements of all cartridges under the effect of back pressure and flow conditioning. Half of the cartridges had similar permeability. Four of them had higher permeability than the average, which is an indication of channeling in the packed bed of the resin whereas one of them had lower permeability, which may be the result of a blocked frit. The variation in permeability values shows that bigger forces are required to disrupt the packed bed.

### Effect of Post Wetting Bed Conditioning by Mechanical Shock/Vibration on the Hydrodynamic Behaviour of Cartridges

The third protocol to pre-treat the cartridges and improve the efficiency test results was based on the post wetting bed conditioning by mechanical shock/vibration. This technique aimed at disrupting the packing of the resin close to the internal walls of the cartridge by knocking it on a hard surface. The dried resin was wetted during the hydrodynamic study of non-conditioned cartridges. An indication that the resin is over compressed and needs to be disrupted close to the internal walls of the cartridge is the tailing in the efficiency tests. Heterogeneity in resin packing structure may be created during the wetting of the dried resin. More specifically during wetting, the resin absorbs water and expands in all directions. The resin can expand in the axial direction, because there is free space between the level of the dried resin and the top internal surface of the cartridge. This is not possible, though, in the radial direction, where there is no free space and as a result, the resin will be compressed more in the radial than in the axial direction during its expansion.

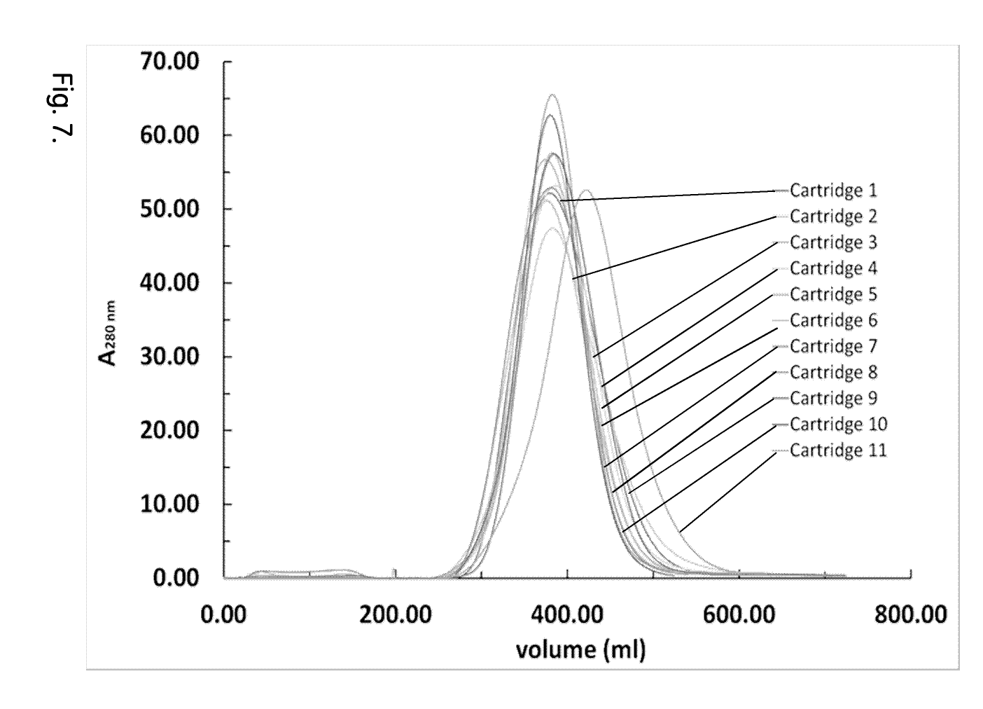

The asymmetry factor of all cartridges was within the range which is required for optimal purification of typical chromatography columns (Table 5). All acetone profiles were free from tailing or secondary peaks (FIG. 7). The acetone profile of cartridge 11 did not overlap with the rest of the acetone profiles, due to resin leakage. Additionally, the acetone peak from the cartridges appeared at around 370 ml, which is close to the packed bed volume of 390 ml (Table 5). This indicates good bed homogeneity and peak symmetry.

TABLE 5
Metrics of the efficiency test under the effect of post wetting bed
conditioning by mechanical shock/vibration

| | Volume at | Peak width at | Asymmetry | | HETP |
|---|---|---|---|---|---|
| Cartridge ID | peak (ml) | 50% (ml) | (10% peak) | N | (μm) |
| 1 | 378.0 | 74.0 | 1.54 | 144.6 | 207.5 |
| 2 | 383.3 | 111.0 | 1.34 | 66.1 | 454.2 |
| 3 | 381.0 | 89.0 | 1.04 | 101.5 | 295.5 |
| 4 | 388.8 | 103.0 | 1.11 | 78.9 | 380.0 |
| 5 | 377.7 | 96.7 | 1.25 | 84.6 | 354.8 |
| 6 | 376.5 | 104.0 | 1.15 | 72.6 | 413.2 |
| 7 | 385.5 | 81.0 | 0.91 | 125.5 | 239.1 |
| 8 | 381.3 | 105.6 | 0.95 | 72.3 | 415.2 |
| 9 | 386.0 | 97.5 | 1.32 | 86.8 | 345.5 |
| 10 | 380.3 | 80.0 | 0.98 | 125.2 | 239.6 |
| 11 | 423.0 | 104.0 | 0.99 | 91.6 | 327.3 |

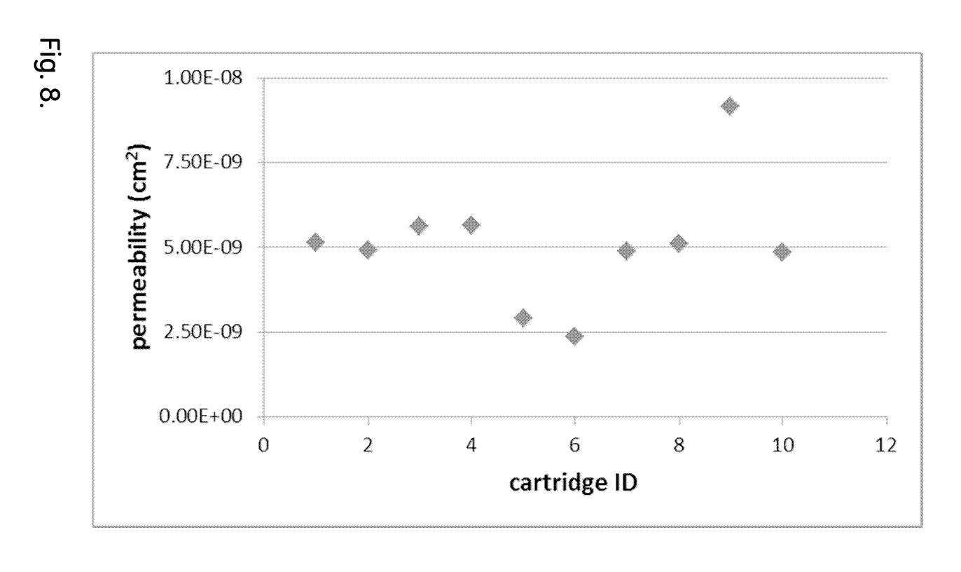

FIG. 8 shows the permeability of cartridges which had undergone post wetting bed conditioning by mechanical shock/vibration. The majority of them had similar permeability except cartridges 5, 6 and 9. Additionally, their values were different from the values obtained under the effect of back pressure and flow conditioning. This shows that the post wetting bed conditioning by mechanical shock/vibration applied once may in some cases give a limited effect. The permeability of cartridge 11 was not estimated due to resin leakage during the application of post wetting bed conditioning by mechanical shock/vibration.

### Effect of Repeated Post Wetting Bed Conditioning by Mechanical Shock/Vibration on the Hydrodynamic Behaviour of Cartridges

The post wetting bed conditioning by mechanical shock/vibration was repeated three times for cartridges 5, 6 and 9 in order to test whether the permeability values of those cartridges would be shifted closer to the average value of the rest of the cartridges. Additionally, it was repeated three times for cartridges 1, 2, 4 and 8 in order to lower their asymmetry factor. The dried resin was wetted during the hydrodynamic study of non-conditioned cartridges. After each repeat the permeability was measured in order to check if its value was closer to the average permeability. Then, efficiency test was run for those cartridges to verify that the metrics of the efficiency test had not been changed. Cartridge 11 has not been tested again due to resin leakage during the development of the previous post-wetting protocol.

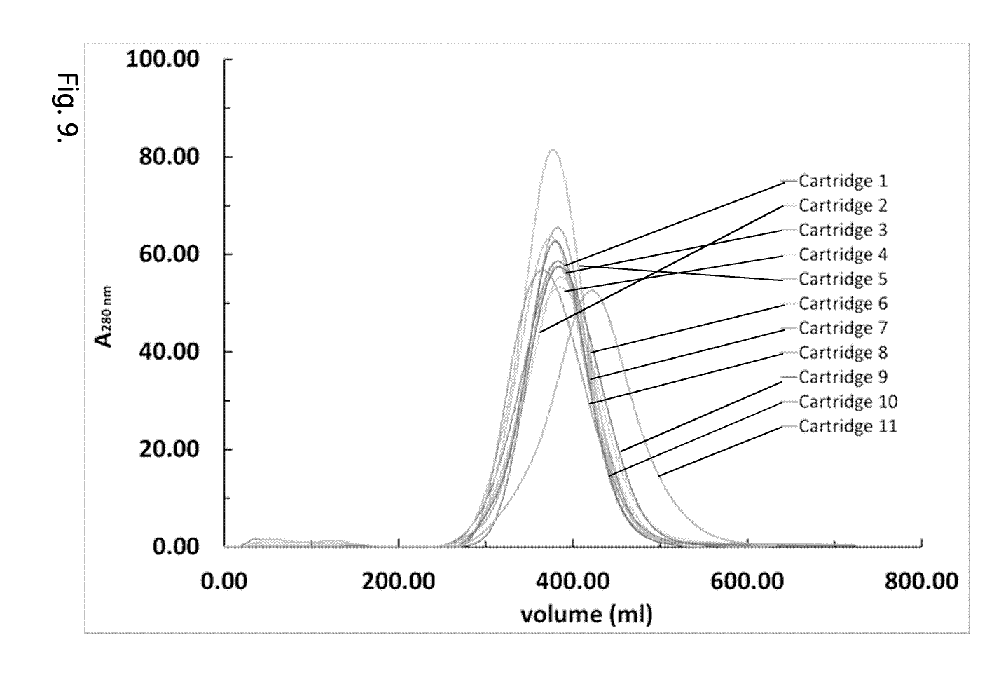

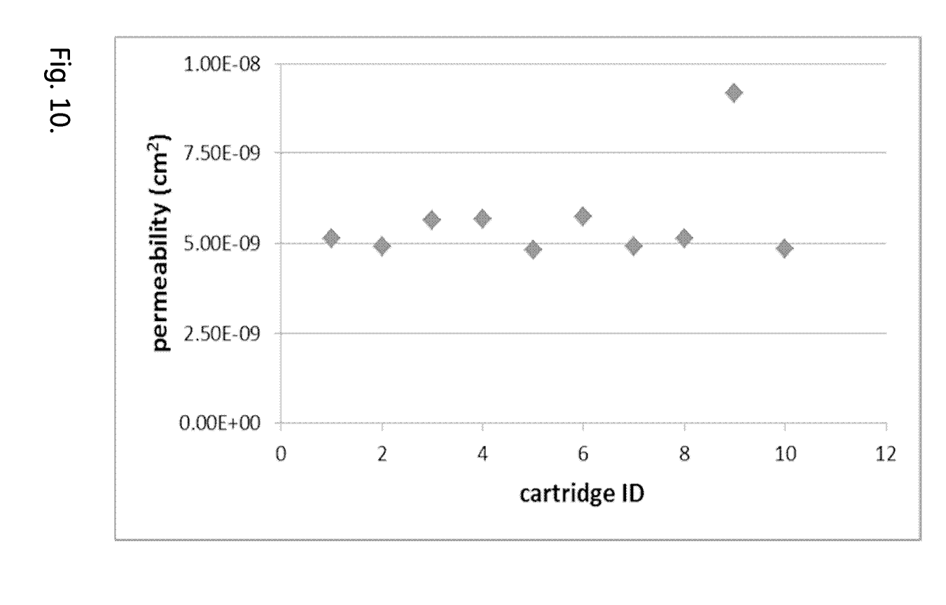

FIG. 9 and Table 6 provide the acetone profiles and the metrics of the acetone tests. FIG. 10 shows the permeability of all cartridges. Repeated post wetting bed conditioning by mechanical shock/vibration was conducted for cartridges 1, 2, 4, 5, 6, 8 and 9. After three repeats of this type of pre-treatment, the permeability of cartridges 5 and 6 was close to the average of the rest of the cartridges. The permeability of cartridges 1, 2, 4 and 8 did not change. No cartridges dripped during efficiency test. All curves had one peak without the presence of tailing and the acetone eluted at 380 ml. Their asymmetry factor was close to one. This was not possible though for cartridge 9. The permeability of this cartridge had not changed. No efficiency test was conducted for cartridge 9 because its permeability was considerably higher than the average permeability.

TABLE 6
Metrics of the efficiency test for cartridges 1, 2, 4, 5, 6 and 8 under the
effect of repeated post wetting bed conditioning by mechanical
shock/vibration.

| | Volume at | Peak width at | Asymmetry | | HETP |
|---|---|---|---|---|---|
| Cartridge ID | peak (ml) | 50% (ml) | (10% peak) | N | (μm) |
| 1 | 383.0 | 95.7 | 0.94 | 88.7 | 338.1 |
| 2 | 386.6 | 89.0 | 0.97 | 104.5 | 287.0 |
| 3 | 381.0 | 89.0 | 1.04 | 101.5 | 295.5 |
| 4 | 381.5 | 103.0 | 1.30 | 76.0 | 394.7 |
| 5 | 377.0 | 82.0 | 1.00 | 117.1 | 256.2 |
| 6 | 375.0 | 88.6 | 1.18 | 99.3 | 302.2 |
| 7 | 385.5 | 81.0 | 0.91 | 125.5 | 239.1 |
| 8 | 365.4 | 101.0 | 1.48 | 72.5 | 413.7 |
| 9 | 386.0 | 97.5 | 1.32 | 86.8 | 345.5 |
| 10 | 380.3 | 80.0 | 0.98 | 125.2 | 239.6 |
| 11 | 423.0 | 104.0 | 0.99 | 91.6 | 327.3 |

### Effect of Repeated Post Wetting Bed Conditioning by Mechanical Shock/Vibration on the Efficiency Test of Parallel Assemblies of Cartridges

Efficiency tests in parallel assemblies of two and four cartridges were conducted to estimate the level of variation in performance. The dried resin was wetted during the hydrodynamic study of non-conditioned cartridges. All cartridges had undergone post wetting bed conditioning by mechanical shock/vibration and they had been flow conditioned as described above. A back pressure of 6 bar was applied downstream in all parallel cartridge assemblies during the efficiency tests. The conditions are shown in Table 1. The combined total volume of the cartridges connected in parallel was 780 ml for the two cartridge assembly and 1570 ml for the four cartridge assembly.

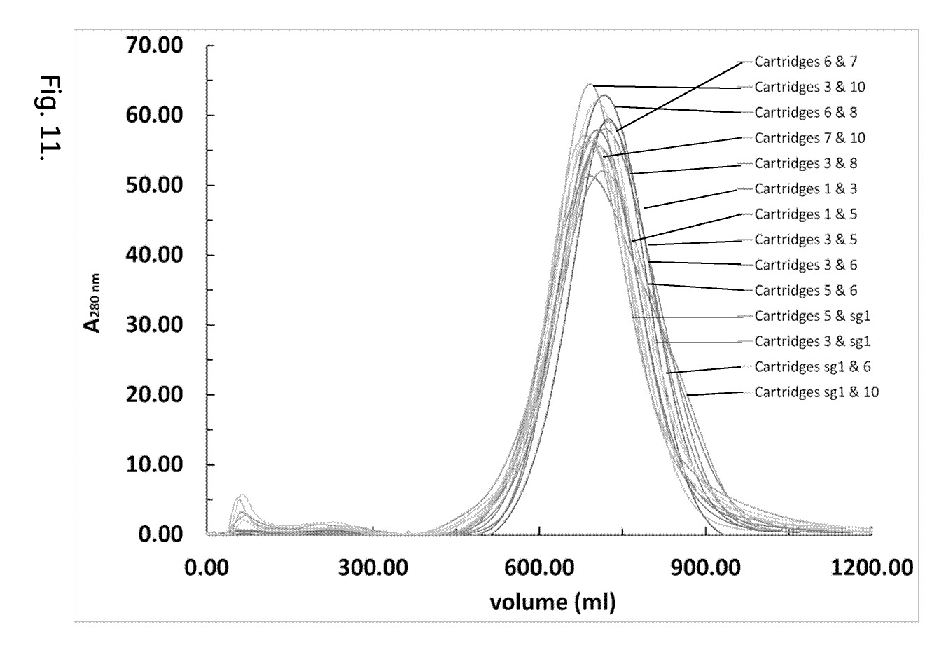

FIG. 11 shows the acetone profiles from the efficiency test of parallel assemblies of two cartridges. There were no leaks during the efficiency tests. Cartridge sg1 was filled with dried resin at a different site and by a different operator. Its permeability was 4.31×10⁻⁹ cm², its asymmetry factor 1.06 and its HETP was 733. The resin was Capto Q and the lot number was 10005939.

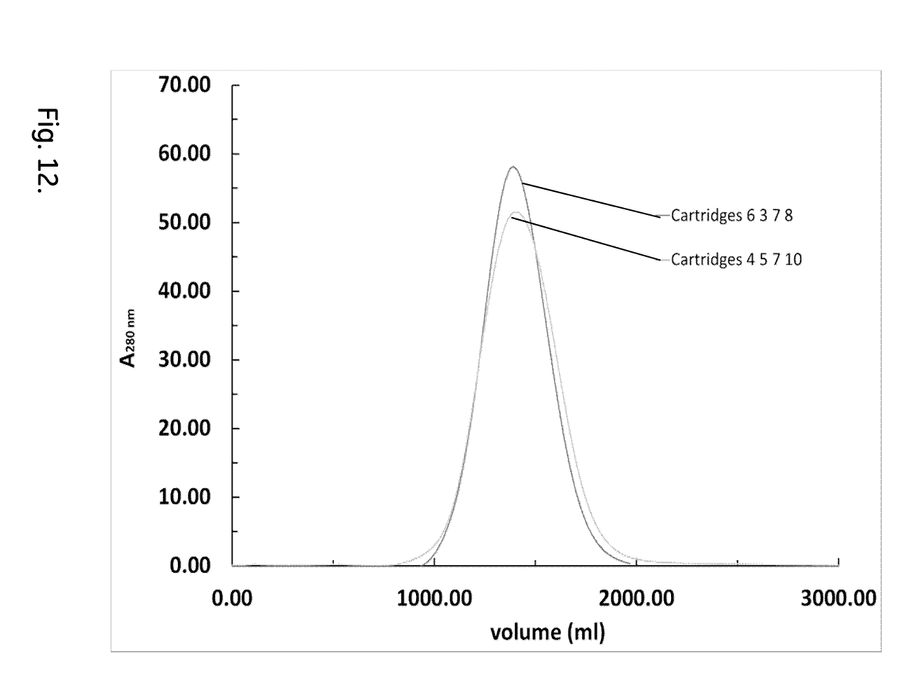

Table 7 contains the main metrics of the efficiency test of the parallel assembly of two cartridges. The asymmetry factor was close to one for all combinations. The acetone peak appeared a little bit earlier than in the case where individual cartridges were used. This is possibly due to the dead volume increase (this refers to the dead volume inside the cartridges). FIG. 12 shows the acetone profiles from the efficiency test of parallel assemblies of four cartridges. Similarly to the case of parallel assembly of two cartridges, there were no leaks during the efficiency tests of parallel assembly of four cartridges with the asymmetry factor to be close one.

TABLE 7
Metrics of the efficiency test for parallel cartridge assemblies

| | Volume at | Peak width at | Asymmetry | | HETP |
|---|---|---|---|---|---|
| Cartridge ID | peak (ml) | 50% (ml) | (10% peak) | N | (μm) |
| 6 & 7 | 705.0 | 182.0 | 1.05 | 83.1 | 361 |
| 3 & 10 | 699.0 | 158.0 | 1.06 | 108.4 | 277 |
| 6 & 8 | 687.0 | 240.0 | 1.55 | 45.4 | 661 |
| 7 & 10 | 682.4 | 195.0 | 1.17 | 67.8 | 442 |
| 3 & 8 | 706.0 | 171.0 | 1.15 | 94.4 | 318 |
| 1 & 3 | 724.0 | 192.0 | 1.11 | 78.8 | 381 |
| 1 & 5 | 717.0 | 226.0 | 1.15 | 55.8 | 538 |
| 3 & 5 | 719.0 | 207.0 | 1.10 | 66.8 | 449 |
| 3 & 6 | 717.0 | 187.0 | 1.13 | 81.4 | 368 |
| 5 & 6 | 725.0 | 162.0 | 0.95 | 111.0 | 270 |
| 5 & sg1 | 689.3 | 169.0 | 1.32 | 92.2 | 326 |
| 3 & sg1 | 710.0 | 191.0 | 1.08 | 76.6 | 392 |
| sg1 & 6 | 704.0 | 177.0 | 1.32 | 87.6 | 342 |
| sg1 & 10 | 704.0 | 162.0 | 1.10 | 104.6 | 287 |
| 6, 3, 7, 8 | 1401.0 | 365.0 | 1.07 | 81.6 | 368 |
| 4, 5, 7, 10 | 1418.0 | 430.0 | 1.08 | 60.2 | 498 |

### Conclusions

The robustness and reproducibility of performance of a new chromatographic modular cartridge design was explored by studying the interplay between resin hydromechanics and chromatographic efficiency. This design was based on a packing method which starts from pre-filled cartridges with dry swellable particles. The cartridge design enables the stacking of two or more cartridges together in a parallel assembly, which provides adjustable capacity and flexibility due to the ease of use of pre-filled cartridges. A protocol has been developed with a sequence of actions to be followed in order to improve the quality of the packing of the resin encased in each cartridge. The results show that cartridge performance in single and parallel assembly in terms of efficiency testing and permeability measurements is quite concise.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties as if individually incorporated.

### Example 2

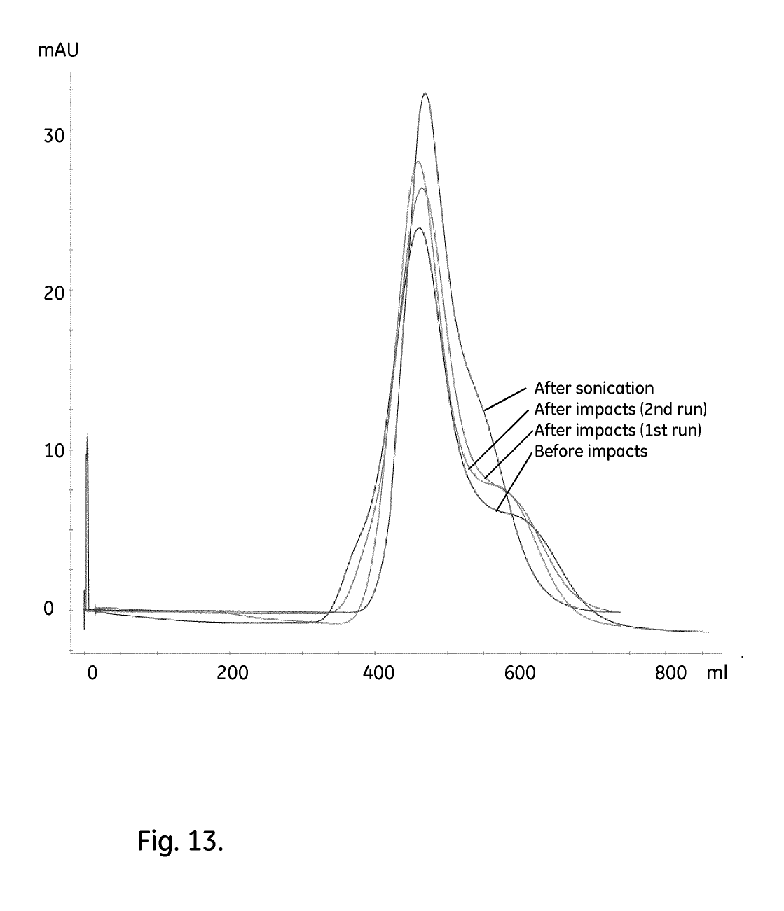

A cartridge was dry-packed similarly to the method described above, with vacuum-dried Capto S ImpAct resin (GE Healthcare Life Sciences) having 50 micrometers average particle diameter when in the swollen state. The cartridge was after swelling conditioned by repeated impacts and also with an additional ultrasound treatment in an Elmasonic S 450H 37 kHz ultrasound bath (Elma Schmidbauer GmbH, Germany). The bath was filled with water and the cartridge was immersed and sonicated for 60 min. Acetone peak profiles (FIG. 13) were run before and after the impacts and the additional sonication. As seen in FIG. 13, the additional sonication caused a considerable improvement of the peak shape.

1. A method for packing a plurality of uniform chromatography columns, comprising the steps of:
a) providing a plurality of chromatography columns;
b) providing a plurality of chromatography resin aliquots;
c) packing said chromatography resin aliquots in said chromatography columns to provide a plurality of packed chromatography columns; and
d) subjecting said packed chromatography columns to repeated mechanical impacts.

2. A method for packing a plurality of uniform chromatography columns, comprising the steps of:
a) providing a plurality of chromatography columns;
b) providing a plurality of chromatography resin aliquots;
c) packing said chromatography resin aliquots in said chromatography columns to provide a plurality of packed chromatography columns; and
d) subjecting said packed chromatography columns to repeated mechanical impacts to provide a plurality of uniform chromatography columns.

3. The method of claim 1, wherein said chromatography resin aliquots are dry and wherein in step c) said chromatography resin aliquots are filled in said chromatography columns and reswollen by adding a liquid to the chromatography columns.

4. The method of claim 1, wherein step b) comprises providing a plurality of dry chromatography resin aliquots and reswelling them with a liquid to provide a plurality of reswollen chromatography resin aliquots, and wherein step c) comprises packing said reswollen chromatography resin aliquots in said chromatography columns.

5. The method of claim 1, wherein in step a) said chromatography resin aliquots are prepared by weighing dry chromatography resin.

6. The method of claim 1, wherein in step a) said chromatography resin aliquots are prepared by weighing dry chromatography resin from a single batch or pool of dry chromatography resin.

7. The method of claim 1, wherein in step a), said chromatography columns are substantially identical.

8. The method of claim 1, wherein said chromatography columns are fixed volume chromatography columns.

9. The method of claim 1, wherein said chromatography columns have bed volumes within the range of 100 mL-25 L.

10. The method of claim 1, wherein said chromatography columns have bed heights within the range of 1-10 cm.

11. The method of claim 1, wherein said chromatography columns have bed heights within the range of 1-5 cm.

12. The method of claim 1, wherein said chromatography columns have bed width to bed height ratios within the range of 2-10.

13. The method of claim 1, wherein said chromatography columns have bed width to bed height ratios within the range of 2-5.

14. The method of claim 1, wherein in step b), said chromatography resin aliquots are substantially identical.

15. The method of claim 1, wherein in step d), said packed chromatography columns are subjected to at least 5 mechanical impacts.

16. The method of claim 1, wherein in step d), said packed chromatography columns are subjected to at least 30 mechanical impacts.

17. The method of claim 15, wherein said mechanical impacts are evenly distributed along the column perimeters.

18. The method of claim 1, wherein in step d), the kinetic energy of said impacts is 0.1-100 J per impact.

19. The method of claim 1, wherein in step d), the total kinetic energy of said impacts is 10-5000 J.

20. The method of claim 1, wherein in step d), said impacts are caused by relative motion between the column and an object.

21. The method of claim 1, wherein in step d), said impacts are caused by vibrating the columns.

22. The method of claim 21, wherein vibrating the columns comprises subjecting them to ultrasound treatment.

23. The method of claim 1, comprising, after step d), a step of subjecting the columns to ultrasound treatment.

24. The method of claim 1, wherein the difference in hydraulic permeability within said plurality of uniform chromatography columns is less than 50%.

25. The method of claim 1, wherein the difference in retention volume for a non-binding species within said plurality of uniform chromatography columns is less than 0.1 column volumes.

26. The method of claim 1, wherein the difference in plate height for a non-binding species within said plurality of uniform chromatography columns is less than 200 micrometers.

27. The method of claim 1, wherein said uniform chromatography columns are single use chromatography columns.

28. The method of claim 1, wherein said plurality of packed chromatography columns is subjected to radiation sterilization after step c), such as before step d).

29. The method of claim 1, wherein said uniform chromatography columns are chromatography column cartridges, adapted to be stacked with like chromatography column cartridges and to be fluidically connected in parallel and/or serially.

30. A method for manufacturing a chromatography apparatus, comprising the steps of:
a) performing the method of claim; and
b) fluidically connecting said plurality of chromatography columns in parallel.

31. The method of claim 30, wherein step b) comprises fluidically connecting said plurality of chromatography columns to a single sample inlet.

32. A method for manufacturing a chromatography apparatus, comprising the steps of:
a) providing a plurality of chromatography columns;
b) providing a plurality of chromatography resin aliquots;
c) packing said chromatography resin aliquots in said chromatography columns to provide a plurality of packed chromatography columns;
d) stacking and fluidically connecting the chromatography columns in parallel to form a chromatography apparatus and
e) subjecting said chromatography apparatus to repeated mechanical impacts.

33. The method of claim 32, further comprising subjecting said chromatography apparatus to radiation sterilization after step d), such as before step e).

34. The method of claim 32, wherein said chromatography columns are fixed volume chromatography columns.
